(19) United States
(12) Patent Application Publication
(10) Pub. No.: US 2025/0205307 A1
(43) Pub. Date: Jun. 26, 2025

(54) Unknown

(71) Applicants: Koji Tanabe, Palo Alto, CA (US); I Peace, Inc., Palo Alto, CA (US)

(72) Inventor: Koji TANABE, Palo Alto, CA (US)

(73) Assignees: Koji Tanabe, Palo Alto, CA (US); I Peace, Inc., Palo Alto, CA (US)

(21) Appl. No.: 18/990,712

(22) Filed: Dec. 20, 2024

(30) Foreign Application Priority Data
Dec. 22, 2023 (JP) 2023-216924

(51) Int. Cl. A61K 38/17; A61K 38/18; A61K 38/19; A61K 38/22; A61K 38/57

(52) U.S. Cl. CPC A61K 38/1709; A61K 38/18; A61K 38/195; A61K 38/22; A61K 38/57

(57) ABSTRACT

A method for decomposing fat, promoting production of prostacyclin or cyclic adenosine monophosphate (cAMP), promoting production of hyaluronic acid in chondrocytes, inducing differentiation of a myoblast to a myotube, and/or treating a lifestyle-related disease or an adult-onset disease, including administering an effective amount of MANF to a subject. A method for inhibiting accumulation of amyloid beta, including administering an effective amount of a composition including MANF, APLN, CXCL4, GDF11, THBS4, and/or TIMP2 to a subject.

### TECHNICAL FIELD

The present invention relates to pharmaceutical compositions and cosmetic compositions.

### BACKGROUND ART

Mesencephalic astrocyte-derived neurotrophic factor (MANF) is a protein identified from a culture medium of rat type-1 astrocyte mesencephalic cells (see, for example, Non-Patent Document 1). MANF protein is conserved across vertebrates and invertebrates. For instance, human MANF protein shares approximately 98% amino acid sequence homology with mouse MANF protein and approximately 50% amino acid sequence homology with MANF proteins of Drosophila and Caenorhabditis elegans (see, for example, Non-Patent Documents 2 and 3).

It is known that expression levels of MANF decrease with aging. Similarly, expression levels of certain proteins other than neurotrophic factors also decrease with aging. For example, growth differentiation factor 11 (GDF11), tissue inhibitor of metalloproteinase-2 (TIMP2), thrombospondin-4 (THBS4), platelet factor 4 (PF4; also known as chemokine C-X-C motif ligand 4 (CXCL4)), and apelin (APLN) are reported to exhibit decreased expression levels with aging (see, for example, Non-Patent Documents 4, 5, 6, and 7). Consequently, associations of aging and anti-aging effects with multiple factors, such as MANF and these proteins, have been suggested (see, for example, Non-Patent Documents 4, 5, 6, and 7).

### PRIOR ART DOCUMENTS

### Non-Patent Documents

[Non-Patent Document 1] Petrova, et al., “MANF: A new mesencephalic astrocyte-derived neurotrophic factor with selectivity for dopaminergic neurons,” Journal of Molecular Neuroscience, 20:173-187 (2003).
[Non-Patent Document 2] Lindholm, et al., “Novel CDNF/MANF family of neurotrophic factors,” Developmental Neurobiology, 70:361-371 (2010).
[Non-Patent Document 3] Lindholm, et al., “MANF is widely expressed in mammalian tissues and differently regulated after ischemic and epileptic insults in rodent brain,” Molecular and Cellular Neuroscience, Vol. 39, Issue 3, 356-371 (2008).
[Non-Patent Document 4] Hsiao, et al., “Role of circulating molecules in age-related cardiovascular and metabolic disorders,” Inflammation and Regeneration, Vol. 42, Issue 2 (2022).
[Non-Patent Document 5] Kang, et al., “Circulating plasma factors involved in rejuvenation,” Aging, Vol. 12, No. 22, 23394-23408 (2020).
[Non-Patent Document 6] Schroer, et al., “Platelet factors attenuate inflammation and rescue cognition in aging,” Nature, Vol. 620, 1071-1079 (2023).
[Non-Patent Document 7] Vinel, et al., “The exerkine apelin reverses age-associated sarcopenia,” Nature Medicine, 24:1360-1371 (2018).

### SUMMARY OF THE INVENTION

### Problems to be Solved by the Invention

One of the objectives of the present invention is to provide pharmaceutical compositions and cosmetic compositions that make effective use of MANF, APLN, GDF11, TIMP2, THBS4, and/or CXCL4.

### Means for Solving the Problem

According to one aspect of the present invention, a fat-decomposing agent, anti-obesity drug, obesity-improving drug, fat decomposition-promoting agent, diet agent, and/or a therapeutic or preventive drug for diabetes containing MANF is/are provided.

According to another aspect of the present invention, a differentiation-promoting agent for inducing differentiation from myoblasts to myotubes, a muscle formation-promoting agent, a muscle mass maintenance agent, and/or a muscle mass-increasing agent containing MANF is/are provided.

According to yet another aspect of the present invention, a prostacyclin production-promoting agent, an adenylate cyclase activator, a cyclic adenosine monophosphate (cAMP) production-promoting agent, a platelet aggregation inhibitor, a vasodilator, a therapeutic or preventive agent for hypertension, a therapeutic or preventive agent for arteriosclerosis, and/or a vascular endothelial cell proliferation inhibitor containing MANF is/are provided.

According to another aspect of the present invention, a hyaluronic acid production-promoting agent for chondrocytes and/or a therapeutic or preventive agent for joint diseases containing MANF is/are provided.

According to another aspect of the present invention, an anti-aging agent containing MANF is provided.

According to another aspect of the present invention, a therapeutic or preventive agent for lifestyle-related diseases containing MANF is provided.

According to another aspect of the present invention, a therapeutic or preventive agent for adult-onset diseases containing MANF is provided.

According to another aspect of the present invention, use of MANF in manufacture of fat-decomposing agents, anti-obesity drugs, obesity-improving agents, fat decomposition-promoting agents, diet agents, and/or therapeutic or preventive drugs for diabetes is provided.

According to another aspect of the present invention, use of MANF in manufacture of differentiation-promoting agents for inducing differentiation from myoblasts to myotubes, muscle formation-promoting agents, muscle mass maintenance agents, and/or muscle mass-increasing agents is provided.

According to another aspect of the present invention, use of MANF in manufacture of prostacyclin production-promoting agents, adenylate cyclase activators, cyclic adenosine monophosphate (cAMP) production-promoting agents, platelet aggregation inhibitors, vasodilators, therapeutic or preventive agents for hypertension, therapeutic or preventive agents for arteriosclerosis, and/or vascular endothelial cell proliferation inhibitors is provided.

According to another aspect of the present invention, use of MANF in manufacture of hyaluronic acid production-promoting agents for chondrocytes and/or therapeutic or preventive agents for joint diseases is provided.

According to another aspect of the present invention, use of MANF in manufacture of anti-aging agents is provided.

According to another aspect of the present invention, use of MANF in manufacture of therapeutic or preventive agents for lifestyle-related diseases is provided.

According to another aspect of the present invention, use of MANF in manufacture of therapeutic or preventive agents for adult-onset diseases is provided.

According to one aspect of the present invention, use of MANF for fat decomposition therapy, anti-obesity therapy, obesity improvement therapy, fat decomposition promotion therapy, diet therapy, and/or treatment or prevention of diabetes is provided.

According to another aspect of the present invention, use of MANF for differentiation-promotion therapy inducing differentiation from myoblasts to myotubes, muscle formation promotion therapy, muscle mass maintenance therapy, and/or muscle mass increase therapy is provided.

According to another aspect of the present invention, use of MANF for prostacyclin production promotion therapy, adenylate cyclase activation therapy, cyclic adenosine monophosphate (cAMP) production promotion therapy, platelet aggregation inhibition therapy, vasodilation therapy, treatment or prevention of hypertension, treatment or prevention of arteriosclerosis, and/or vascular endothelial cell proliferation inhibition therapy is provided.

According to another aspect of the present invention, use of MANF for hyaluronic acid production promotion therapy in chondrocytes and/or treatment or prevention of joint diseases is provided.

According to another aspect of the present invention, use of MANF for anti-aging therapy is provided.

According to another aspect of the present invention, use of MANF for treatment or prevention of lifestyle-related diseases is provided.

According to another aspect of the present invention, use of MANF for treatment or prevention of adult-onset diseases is provided.

According to another aspect of the present invention, methods for fat decomposition therapy, anti-obesity therapy, obesity improvement therapy, fat decomposition promotion therapy, diet therapy, and/or treatment or prevention of diabetes, which include administering a composition including MANF to a subject, are provided. The subject may be a human or non-human animal, or their cells.

According to another aspect of the present invention, methods for differentiation-promotion therapy inducing differentiation from myoblasts to myotubes, muscle formation promotion therapy, muscle mass maintenance therapy, and/or muscle mass increase therapy, which include administering a composition including MANF to a subject, are provided.

According to another aspect of the present invention, methods for prostacyclin production promotion therapy, adenylate cyclase activation therapy, cyclic adenosine monophosphate (cAMP) production promotion therapy, platelet aggregation inhibition therapy, vasodilation therapy, treatment or prevention of hypertension, treatment or prevention of arteriosclerosis, and/or vascular endothelial cell proliferation inhibition therapy, which include administering a composition including MANF to a subject, are provided.

According to another aspect of the present invention, methods for hyaluronic acid production promotion therapy in chondrocytes and/or treatment or prevention of joint diseases, which include administering a composition including MANF to a subject, are provided.

According to another aspect of the present invention, methods for anti-aging therapy, which include administering a composition including MANF to a subject, are provided.

According to another aspect of the present invention, methods for treatment or prevention of lifestyle-related diseases, which include administering a composition including MANF to a subject, are provided.

According to another aspect of the present invention, methods for treatment or prevention of adult-onset diseases, which include administering a composition including MANF to a subject, are provided.

According to another aspect of the present invention, inhibitors of protein accumulation, aggregation, or mislocalization, preferably amyloid-beta inhibitors, including MANF, APLN, CXCL4, GDF11, THBS4, and/or TIMP2, are provided.

According to another aspect of the present invention, inhibitors of neuronal cell death including MANF, APLN, CXCL4, GDF11, THBS4, and/or TIMP2, are provided.

According to another aspect of the present invention, therapeutic or preventive agents for neurological diseases including MANF, APLN, CXCL4, GDF11, THBS4, and/or TIMP2, are provided. The neurological diseases in this aspect may include diseases associated with protein accumulation, aggregation, or mislocalization, such as amyloid beta, preferably those caused by accumulation or aggregation of amyloid beta. The neurological diseases may also include neurofibrillary tangles (NFT).

According to another aspect of the present invention, therapeutic or preventive agents for diseases caused by protein accumulation, aggregation, or mislocalization, including MANF, APLN, CXCL4, GDF11, THBS4, and/or TIMP2, are provided. The proteins in this aspect may include, for example, amyloid beta, and the diseases may include, for example, amyloidosis.

According to another aspect of the present invention, therapeutic or preventive agents for dementia including MANF, APLN, CXCL4, GDF11, THBS4, and/or TIMP2, are provided. The dementia in this aspect may include Alzheimer's disease and/or Lewy body dementia, as well as dementia associated with diabetes patients and/or Down syndrome patients.

According to another aspect of the present invention, therapeutic or preventive agents for diseases caused by neuronal cell death including MANF, APLN, CXCL4, GDF11, THBS4, and/or TIMP2, are provided. The diseases in this aspect may include Alzheimer's disease, Huntington's disease, Parkinson's disease, and/or amyotrophic lateral sclerosis (ALS).

According to one aspect of the present invention, therapeutic or preventive agents for treatment or prevention of neuronal cell loss or neuronal cell death, including MANF, APLN, CXCL4, GDF11, THBS4, and/or TIMP2, are provided.

According to another aspect of the present invention, rejuvenation or anti-aging agents for neurons, including MANF, APLN, CXCL4, GDF11, THBS4, and/or TIMP2, are provided.

According to another aspect of the present invention, use of MANF, APLN, CXCL4, GDF11, THBS4, and/or TIMP2 in manufacture of inhibitors of protein accumulation, aggregation, or mislocalization, preferably amyloid-beta inhibitors, is provided.

According to another aspect of the present invention, use of MANF, APLN, CXCL4, GDF11, THBS4, and/or TIMP2 in manufacture of inhibitors of neuronal cell death is provided.

According to another aspect of the present invention, use of MANF, APLN, CXCL4, GDF11, THBS4, and/or TIMP2 in manufacture of therapeutic or preventive agents for neurological diseases is provided. The neurological diseases of this aspect may include diseases associated with protein accumulation, aggregation, or mislocalization, such as amyloid beta, preferably neurological diseases caused by accumulation or aggregation of amyloid beta. These neurological diseases may include neurofibrillary tangles (NFT).

According to another aspect of the present invention, use of MANF, APLN, CXCL4, GDF11, THBS4, and/or TIMP2 in manufacture of therapeutic or preventive agents for diseases caused by protein accumulation, aggregation, or mislocalization is provided. The proteins in this aspect may include, for example, amyloid beta, and the diseases may include, for example, amyloidosis.

According to another aspect of the present invention, use of MANF, APLN, CXCL4, GDF11, THBS4, and/or TIMP2 in manufacture of therapeutic or preventive agents for dementia is provided.

The dementia in this aspect may include Alzheimer's disease and/or Lewy body dementia, as well as dementia associated with diabetes or Down syndrome.

According to another aspect of the present invention, use of MANF, APLN, CXCL4, GDF11, THBS4, and/or TIMP2 in manufacture of therapeutic or preventive agents for diseases caused by neuronal cell death is provided. These diseases may include Alzheimer's disease, Huntington's disease, Parkinson's disease, and/or amyotrophic lateral sclerosis (ALS).

According to another aspect of the present invention, use of MANF, APLN, CXCL4, GDF11, THBS4, and/or TIMP2 in manufacture of therapeutic or preventive agents for treatment or prevention of neuronal cell loss or neuronal cell death is provided.

According to another aspect of the present invention, use of MANF, APLN, CXCL4, GDF11, THBS4, and/or TIMP2 in manufacture of rejuvenation or anti-aging agents for neurons is provided.

According to another aspect of the present invention, use of MANF, APLN, CXCL4, GDF11, THBS4, and/or TIMP2 for inhibiting protein accumulation, aggregation, or mislocalization, preferably amyloid-beta inhibition, is provided.

According to another aspect of the present invention, use of MANF, APLN, CXCL4, GDF11, THBS4, and/or TIMP2 for inhibiting neuronal cell death is provided.

According to another aspect of the present invention, use of MANF, APLN, CXCL4, GDF11, THBS4, and/or TIMP2 for treatment or prevention of neurological diseases is provided. These neurological diseases may include diseases associated with protein accumulation, aggregation, or mislocalization, such as amyloid beta, preferably neurological diseases caused by the accumulation or aggregation of amyloid beta. These diseases may also include neurofibrillary tangles (NFT).

According to another aspect of the present invention, use of MANF, APLN, CXCL4, GDF11, THBS4, and/or TIMP2 for treatment or prevention of diseases caused by protein accumulation, aggregation, or mislocalization is provided. The proteins in this aspect may include amyloid beta, and the diseases may include amyloidosis.

According to another aspect of the present invention, use of MANF, APLN, CXCL4, GDF11, THBS4, and/or TIMP2 for treatment or prevention of dementia is provided. The dementia in this aspect may include Alzheimer's disease and/or Lewy body dementia, as well as dementia associated with diabetes or Down syndrome.

According to another aspect of the present invention, use of MANF, APLN, CXCL4, GDF11, THBS4, and/or TIMP2 for treatment or prevention of diseases caused by neuronal cell death is provided. These diseases may include Alzheimer's disease, Huntington's disease, Parkinson's disease, and/or amyotrophic lateral sclerosis (ALS).

According to another aspect of the present invention, use of MANF, APLN, CXCL4, GDF11, THBS4, and/or TIMP2 for treatment or prevention of neuronal cell loss or neuronal cell death is provided.

According to another aspect of the present invention, use of MANF, APLN, CXCL4, GDF11, THBS4, and/or TIMP2 for rejuvenation or anti-aging of neurons is provided.

According to another aspect of the present invention, methods for inhibiting protein accumulation, aggregation, or mislocalization, preferably amyloid-beta inhibition, comprising administering a composition containing MANF, APLN, CXCL4, GDF11, THBS4, and/or TIMP2 to a subject, are provided.

According to another aspect of the present invention, methods for inhibiting neuronal cell death comprising administering a composition containing MANF, APLN, CXCL4, GDF11, THBS4, and/or TIMP2 to a subject, are provided.

According to one aspect of the present invention, a method for treatment or prevention of neurological diseases, comprising administering a composition containing MANF, APLN, CXCL4, GDF11, THBS4, and/or TIMP2 to a subject, is provided. The neurological diseases of this aspect may include diseases associated with the accumulation, aggregation, or mislocalization of proteins such as amyloid beta, preferably neurological diseases caused by the accumulation or aggregation of amyloid beta. These diseases may also include neurofibrillary tangles (NFT).

According to another aspect of the present invention, a method for treatment or prevention of diseases caused by accumulation, aggregation, or mislocalization of proteins, comprising administering a composition containing MANF, APLN, CXCL4, GDF11, THBS4, and/or TIMP2 to a subject, is provided. The proteins in this aspect may include, for example, amyloid beta, and the diseases may include, for example, amyloidosis.

According to another aspect of the present invention, a method for treatment or prevention of dementia, comprising administering a composition containing MANF, APLN, CXCL4, GDF11, THBS4, and/or TIMP2 to a subject, is provided. The dementia in this aspect may include Alzheimer's disease and/or Lewy body dementia, as well as dementia associated with diabetes or Down syndrome.

According to another aspect of the present invention, a method for treatment or prevention of diseases caused by neuronal cell death, comprising administering a composition containing MANF, APLN, CXCL4, GDF11, THBS4, and/or TIMP2 to a subject, is provided. These diseases may include Alzheimer's disease, Huntington's disease, Parkinson's disease, and/or amyotrophic lateral sclerosis (ALS).

According to another aspect of the present invention, a method for treatment or prevention of neuronal cell loss or neuronal cell death, comprising administering a composition containing MANF, APLN, CXCL4, GDF11, THBS4, and/or TIMP2 to a subject, is provided.

According to another aspect of the present invention, a method for the rejuvenation or anti-aging of neurons, comprising administering a composition containing MANF, APLN, CXCL4, GDF11, THBS4, and/or TIMP2 to a subject, is provided.

### Effect of the Invention

According to the present invention, it is possible to provide pharmaceutical compositions and cosmetic compositions that effectively utilize MANF, APLN, GDF11, TIMP2, THBS4, and/or CXCL4.

### BRIEF DESCRIPTION OF THE DRAWINGS

FIG. 1 A photograph of adipocytes according to Example 2.

FIG. 2 A table and graph showing the measurement results of free glycerol when MANF and isoproterenol were added to the culture medium of adipocytes in Example 2.

FIG. 3 A photograph of cells stained with an anti-MHC antibody according to Example 3.

FIG. 4 A table and graph showing the measurement results of the differentiation index into myotubes when MANF and insulin were added to the culture medium of normal human skeletal muscle myoblasts in Example 3.

FIG. 5 A table and graph showing the measurement results of the amount of 6-keto-prostaglandin Fla when MANF and arachidonic acid were added to the culture medium of normal human umbilical vein endothelial cells in Example 4.

FIG. 6 A table and graph showing the measurement results of hyaluronic acid production when MANF and N-acetylglucosamine were added to the culture medium of human chondrocytes and cultured for 24 hours in Example 5.

FIG. 7 A table and graph showing the measurement results of hyaluronic acid production when MANF and N-acetylglucosamine were added to the culture medium of human chondrocytes and cultured for 72 hours in Example 5.

FIG. 8 A photograph of cerebral neurons according to Example 7.

FIG. 9 A table and graph showing the number of surviving cerebral neurons per field when cultured with amyloid beta alone, with amyloid beta and one of GDF11, TIMP2, THBS4, or CXCL4, or without any addition in Example 7.

FIG. 10 A photograph of cerebral neurons according to Example 8. The scale bar in the figure represents 150 μm.

FIG. 11 A table and graph showing the number of surviving cerebral neurons per field when cultured with amyloid beta alone, with amyloid beta and one of MANF or APLN, or without any addition in Example 8.

FIG. 12 A table and graph showing the number of Cleaved Caspase-3 positive cerebral neurons per field according to Example 9.

### DESCRIPTION OF EMBODIMENTS

The embodiments of the present invention will now be described in detail. The embodiments presented below exemplify devices or methods for embodying the technical idea of the invention, but the technical idea of the present invention is not limited to the combinations of components described herein. Various modifications can be made to the technical idea of the invention within the scope of the claims.

### First Embodiment

The pharmaceutical composition and/or cosmetic composition according to the first embodiment includes MANF.

The MANF included in the pharmaceutical composition and/or cosmetic composition of the first embodiment may be, for example, an isolated protein or recombinant protein, preferably a recombinant human MANF protein. The MANF protein may have, for example, the amino acid sequence of SEQ ID NO: 1, or an amino acid sequence having at least 50%, 60%, 65%, 70%, 75%, 80%, 85%, 90%, 95%, 98%, or 99% homology to the amino acid sequence of SEQ ID NO: 1. The MANF protein may also have the amino acid sequence (SEQ ID NO: 2) of positions 25 to 182 in SEQ ID NO: 1 or an amino acid sequence having at least 60%, 65%, 70%, 75%, 80%, 85%, 90%, 95%, 98%, or 99% homology to SEQ ID NO: 2.

The pharmaceutical composition and/or cosmetic composition containing MANF in the first embodiment can be used as, for example, a fat decomposition agent, anti-obesity drug, obesity improvement agent, fat decomposition promoter, diet agent, therapeutic or preventive agent for diabetes, differentiation promoter for inducing differentiation from myoblasts to myotubes, muscle formation promoter, muscle mass maintenance agent, muscle mass increasing agent, prostacyclin production promoter, adenylate cyclase activator, cyclic adenosine monophosphate (cAMP) production promoter, platelet aggregation inhibitor, vasodilator, therapeutic or preventive agent for hypertension, therapeutic or preventive agent for arteriosclerosis, vascular endothelial cell proliferation inhibitor, hyaluronic acid production promoter in chondrocytes, therapeutic or preventive agent for joint diseases, anti-aging agent, therapeutic or preventive agent for lifestyle-related diseases, and/or therapeutic or preventive agent for adult diseases.

The inventors of the present invention have discovered for the first time that MANF decomposes fat. Accordingly, MANF can be used as a fat decomposition agent, anti-obesity drug, obesity improvement agent, fat decomposition promoter, and diet agent. Additionally, it is known that fat levels in the blood increase in diabetes. Therefore, MANF can be used as a therapeutic or preventive agent for diabetes. The fat may include, for example, triglycerides. Diabetes may include type 1 diabetes, type 2 diabetes, diabetes caused by other specific mechanisms or diseases, and gestational diabetes.

The inventors of the present invention have also discovered for the first time that MANF promotes differentiation of myoblasts into myotubes. Thus, MANF can be used as a differentiation promoter for inducing differentiation from myoblasts to myotubes. Since myotubes form muscle fibers and eventually become muscles, MANF can also be used as a muscle formation promoter, muscle mass maintenance agent, and muscle mass increasing agent.

The inventors of the present invention have also discovered for the first time that MANF promotes prostacyclin production in endothelial cells. Therefore, MANF can be used as a prostacyclin production promoter. Prostacyclin, also known as prostaglandin I2 (PGI2), activates adenylate cyclase and promotes the production of cyclic adenosine monophosphate (cAMP). Consequently, MANF can be used as an adenylate cyclase activator and cAMP production promoter.

Moreover, prostacyclin inhibits platelet aggregation and dilates vascular smooth muscles. Therefore, MANF can be used as a platelet aggregation inhibitor, vasodilator, therapeutic or preventive agent for hypertension, and therapeutic or preventive agent for arteriosclerosis. Additionally, prostacyclin inhibits vascular endothelial cell proliferation, making MANF applicable as a vascular endothelial cell growth inhibitor.

The inventors of the present invention have also discovered for the first time that MANF promotes hyaluronic acid production in chondrocytes. Therefore, MANF can be used as a hyaluronic acid production promoter for chondrocytes. Hyaluronic acid protects cartilage and has lubricating, cushioning, and anti-inflammatory effects on joints. Accordingly, MANF can be used as a therapeutic or preventive agent for joint diseases. Joint diseases may include arthritis, osteoarthritis, osteoarthrosis of the knee, osteoarthrosis of the hip, lumbar osteoarthritis, meniscus injuries, rheumatoid arthritis, ligament injuries of the knee, jumper's knee (patellar tendinitis), runner's knee (iliotibial band syndrome), pes anserinus bursitis, Baker's cyst, osteochondritis dissecans, patellofemoral arthritis, Hoffa's disease, and others. MANF can also be used to treat or prevent joint pain associated with these joint diseases.

Anti-obesity effects, muscle mass-increasing effects, vasodilating effects, and cartilage-protecting effects may suppress aging, for example, by protecting cells from cytotoxic stress and endoplasmic reticulum (ER) stress, leading to the treatment or prevention of lifestyle-related diseases and adult-onset diseases. Thus, MANF can be used as an anti-aging agent, therapeutic or preventive agent for lifestyle-related diseases, and/or therapeutic or preventive agent for adult-onset diseases.

The pharmaceutical composition and cosmetic composition of the first embodiment contain MANF in an effective amount. An “effective amount” refers to the amount sufficient to exert the intended therapeutic or cosmetic effect. The effective amount can be appropriately determined depending on factors such as the patient's age, the target disease, the presence of other active ingredients, and the amount of other excipients.

The pharmaceutical composition and/or cosmetic composition of the first embodiment may further include pharmaceutically acceptable carriers, excipients, disintegrants, buffers, emulsifiers, suspending agents, stabilizers, preservatives, and physiological saline. Examples of excipients include lactose, starch, sorbitol, D-mannitol, and sucrose. Examples of disintegrants include carboxymethylcellulose and calcium carbonate. Examples of buffers include phosphates, citrates, and acetates. Examples of emulsifiers include gum arabic, sodium alginate, and tragacanth.

Examples of suspending agents include glyceryl monostearate, aluminum monostearate, methylcellulose, carboxymethylcellulose, hydroxymethylcellulose, and sodium lauryl sulfate. Examples of anesthetics include benzyl alcohol, chlorobutanol, and sorbitol. Examples of stabilizers include propylene glycol and ascorbic acid. Examples of preservatives include phenol, benzalkonium chloride, benzyl alcohol, chlorobutanol, and methylparaben. Examples of antimicrobials include benzalkonium chloride, paraoxybenzoic acid, and chlorobutanol.

The pharmaceutical composition and/or cosmetic composition according to the first embodiment may further include water, alcohol, surfactants (cationic, anionic, nonionic, and amphoteric surfactants), humectants (glycerin, 1,3-butyleneglycol, propylene glycol, propanediol, pentanediol, polyquaternium, amino acids, urea, pyrrolidone carboxylic acid salts, nucleotides, monosaccharides, oligosaccharides, and their derivatives), thickeners (polysaccharides, polyacrylate salts, carboxyvinyl polymers, polyvinylpyrrolidone, polyvinyl alcohol, chitin, chitosan, alginate, carrageenan, xanthan gum, methylcellulose, and their derivatives), waxes, petrolatum, saturated hydrocarbons, unsaturated fatty acids, silicone oils, and their derivatives, triglycerides such as tri(caprylyl/caprin)glyceride and trioctanoin, ester oils such as isopropyl stearate, natural oils and fats (olive oil, camellia oil, avocado oil, almond oil, cocoa butter, evening primrose oil, grape seed oil, macadamia nut oil, eucalyptus oil, rosehip oil, squalane, orange roughy oil, lanolin, and ceramides), preservatives (oxybenzoic acid derivatives, dehydroacetic acid salts, photosensitizers, sorbic acid, and phenoxyethanol), bactericides (sulfur, triclocarbanilide, salicylic acid, zinc pyrithione, hinokitiol, and their derivatives), UV absorbers (para-aminobenzoic acid, methoxycinnamic acid, and their derivatives), anti-inflammatory agents (allantoin, bisabolol, ε-aminocaproic acid, acetyl farnesyl cysteine, glycyrrhetinic acid, and their derivatives), antioxidants (tocopherols, BHA, BHT, astaxanthin, and their derivatives), chelating agents (edetic acid, hydroxyethane diphosphonic acid, and their derivatives), plant and animal extracts (Angelica keiskei, aloe, jujube, Scutellaria, Phellodendron, seaweed, quince, chamomile, licorice, kiwi, cucumber, mulberry, birch, angelica, garlic, peony, hops, horse chestnut, lavender, rosemary, eucalyptus, milk, various peptides, placenta, royal jelly, Euglena extracts, hydrolyzed Euglena extracts, and Euglena oil), pH adjusters (inorganic acids, inorganic acid salts, organic acids, organic acid salts, and their derivatives), vitamins (vitamin A, B, C, D, ubiquinone, nicotinamide, and their derivatives), yeast, koji mold and lactic acid bacteria fermentation liquids, Galactomyces fermentation liquids, whitening agents (tranexamic acid, tranexamic acid cetyl hydrochloride, 4-n-butylresorcinol, arbutin, kojic acid, ellagic acid, licorice flavonoids, niacinamide, and vitamin C derivatives), ceramides and ceramide derivatives, anti-wrinkle agents (retinol, retinal, their derivatives, nicotinamide, oligopeptides, and their derivatives), and powders such as titanium oxide, talc, mica, silica, zinc oxide, iron oxide, and processed forms of these powders. These may be formulated to achieve the objectives of the pharmaceutical and cosmetic compositions in this embodiment.

The ingredients that can be added to the pharmaceutical composition and/or cosmetic composition of the first embodiment are not limited to the above examples. Any ingredient suitable for use in pharmaceutical and/or cosmetic compositions may be freely selected. When using the composition as a poultice, in addition to the above components, bases (e.g., kaolin, bentonite) and gelling agents (e.g., polyacrylate salts, polyvinyl alcohol) may be added within the scope of achieving the intended purpose. When using the composition as a bath additive, sulfates, bicarbonates, borates, dyes, and humectants may be appropriately added and prepared as powder or liquid formulations. The pharmaceutical composition and/or cosmetic composition of the first embodiment may also be a topical skin composition.

Another aspect of the first embodiment is the use of MANF in manufacture of pharmaceutical compositions and/or cosmetic compositions. Such compositions may include, for example, fat decomposition agents, anti-obesity drugs, obesity improvement agents, fat decomposition promoters, diet agents, therapeutic or preventive agents for diabetes, differentiation promoters for inducing differentiation from myoblasts to myotubes, muscle formation promoters, muscle mass maintenance agents, muscle mass increasing agents, prostacyclin production promoters, adenylate cyclase activators, cyclic adenosine monophosphate (cAMP) production promoters, platelet aggregation inhibitors, vasodilators, therapeutic or preventive agents for hypertension, therapeutic or preventive agents for arteriosclerosis, vascular endothelial cell growth inhibitors, hyaluronic acid production promoters in chondrocytes, therapeutic or preventive agents for joint diseases, anti-aging agents, therapeutic or preventive agents for lifestyle-related diseases, and/or therapeutic or preventive agents for adult diseases.

In this aspect, the process of manufacturing pharmaceutical compositions and/or cosmetic compositions may include a step of adding MANF, without limitations on the timing or method of addition. This aspect may include a step of mixing MANF with pharmaceutically acceptable carriers, excipients, disintegrants, buffers, emulsifiers, suspending agents, anesthetics, stabilizers, preservatives, antimicrobial agents, and physiological saline, or with water, alcohol, surfactants, humectants, thickeners, waxes, petrolatum, saturated hydrocarbons, unsaturated fatty acids, silicone oils, and their derivatives, triglycerides, ester oils, natural oils and fats, preservatives, bactericides, UV absorbers, anti-inflammatory agents, antioxidants, chelating agents, plant and animal extracts, pH adjusters, vitamins, fermentation liquids from yeast, koji mold or lactic acid bacteria, Galactomyces culture fluids, whitening agents, ceramides and ceramide derivatives, anti-wrinkle agents, titanium oxide, talc, mica, silica, zinc oxide, iron oxide, silicone, and processed powders thereof.

Another aspect of the first embodiment is a method of administering a pharmaceutical composition and/or cosmetic composition containing MANF to a subject. This aspect may include, for example, methods for fat decomposition therapy, obesity treatment therapy, obesity improvement therapy, fat decomposition promotion therapy, diet therapy, treatment or prevention of diabetes, differentiation promotion therapy for inducing differentiation from myoblasts to myotubes, muscle formation promotion therapy, muscle mass maintenance therapy, muscle mass increase therapy, prostacyclin production promotion therapy, adenylate cyclase activation therapy, cAMP production promotion therapy, platelet aggregation inhibition therapy, vasodilation therapy, treatment or prevention of hypertension, treatment or prevention of arteriosclerosis, vascular endothelial cell growth inhibition therapy, hyaluronic acid production promotion therapy in chondrocytes, treatment or prevention of joint diseases, anti-aging therapy, treatment or prevention of lifestyle-related diseases, and/or treatment or prevention of adult diseases.

In the method according to the first embodiment, the pharmaceutical composition and/or cosmetic composition containing MANF is administered to a subject. The subject may be a human or a non-human animal, or their cells.

In this specification, “administration” includes delivery of a pharmaceutical composition and/or cosmetic composition to a subject to provide the active ingredient. This may involve oral administration, topical administration, intravenous administration, intraperitoneal administration, intramuscular administration, or subcutaneous administration. The method of administration is not limited to these methods and may involve combinations thereof. For example, when the pharmaceutical or cosmetic composition is prepared as a powder or liquid, the composition may be administered orally, topically, intravenously, intraperitoneally, intramuscularly, or subcutaneously. Additionally, when the composition is a poultice or topical skin application, it may be administered transdermally through local contact. If the subject consists of cells derived from humans or non-human animals, “administration” may also involve contacting the cells with a medium containing the pharmaceutical or cosmetic composition. Thus, administration may be conducted in vivo or in vitro.

In the method according to the first embodiment, the pharmaceutical composition and/or cosmetic composition containing MANF is administered to the subject in an effective amount. Here, “effective amount” refers to an amount sufficient to achieve the intended effect as a pharmaceutical or cosmetic composition. The effective amount may be appropriately set based on factors such as the subject's age, the condition being treated, the purpose of administration, the presence of other active ingredients, and the quantity of other components.

In the method according to the first embodiment, the frequency, number of doses, and duration of administering the pharmaceutical composition and/or cosmetic composition containing MANF are not limited and may be appropriately adjusted to deliver an effective amount of MANF to the subject. For example, the frequency of administration may be twice daily, once daily, every other day, or once every three days.

### Second Embodiment

The pharmaceutical composition and/or cosmetic composition according to the second embodiment comprises MANF, APLN, CXCL4, GDF11, THBS4, and/or TIMP2. The pharmaceutical composition and/or cosmetic composition may include one selected from CXCL4, GDF11, THBS4, and TIMP2, or two or more thereof.

The CXCL4 included in the pharmaceutical composition and/or cosmetic composition according to the second embodiment may be an isolated protein or a recombinant protein, preferably a recombinant human CXCL4 protein. The CXCL4 protein may have, for example, the amino acid sequence of SEQ ID NO: 3, or an amino acid sequence having at least 50%, 60%, 65%, 70%, 75%, 80%, 85%, 90%, 95%, 98%, or 99% homology to SEQ ID NO: 3. The CXCL4 protein may also have the amino acid sequence (SEQ ID NO: 4) of positions 32 to 101 in SEQ ID NO: 3, or an amino acid sequence having at least 60%, 65%, 70%, 75%, 80%, 85%, 90%, 95%, 98%, or 99% homology to SEQ ID NO: 4.

The GDF11 included in the pharmaceutical composition and/or cosmetic composition according to the second embodiment may be an isolated protein or a recombinant protein, preferably a recombinant human GDF11 protein. The GDF11 protein may have, for example, the amino acid sequence of SEQ ID NO: 5, or an amino acid sequence having at least 50%, 60%, 65%, 70%, 75%, 80%, 85%, 90%, 95%, 98%, or 99% homology to SEQ ID NO: 5. The GDF11 protein may also have the amino acid sequence (SEQ ID NO: 6) of positions 299 to 407 in SEQ ID NO: 5, or an amino acid sequence having at least 60%, 65%, 70%, 75%, 80%, 85%, 90%, 95%, 98%, or 99% homology to SEQ ID NO: 6.

The TIMP2 included in the pharmaceutical composition and/or cosmetic composition according to the second embodiment may be an isolated protein or a recombinant protein, preferably a recombinant human TIMP2 protein. The TIMP2 protein may have, for example, the amino acid sequence of SEQ ID NO: 7, or an amino acid sequence having at least 50%, 60%, 65%, 70%, 75%, 80%, 85%, 90%, 95%, 98%, or 99% homology to SEQ ID NO: 7. The TIMP2 protein may also have the amino acid sequence (SEQ ID NO: 8) of positions 27 to 220 in SEQ ID NO: 7, or an amino acid sequence having at least 60%, 65%, 70%, 75%, 80%, 85%, 90%, 95%, 98%, or 99% homology to SEQ ID NO: 8.

The THBS4 included in the pharmaceutical composition and/or cosmetic composition according to the second embodiment may be an isolated protein or a recombinant protein, preferably a recombinant human THBS4 protein. The THBS4 protein may have, for example, the amino acid sequence of SEQ ID NO: 9, or an amino acid sequence having at least 50%, 60%, 65%, 70%, 75%, 80%, 85%, 90%, 95%, 98%, or 99% homology to SEQ ID NO: 9.

The APLN included in the pharmaceutical composition and/or cosmetic composition according to the second embodiment is preferably Apelin-13. Apelin is a protein precursor composed of 77 amino acid residues, which is cleaved into Apelin-36 (36 amino acids), Apelin-17 (17 amino acids), and Apelin-13 (13 amino acids). Among these, Apelin-13 exhibits the highest activity. APLN, preferably Apelin-13, may have, for example, the amino acid sequence of SEQ ID NO: 10 (XRPRLSHKGPMPF, where X is pyroglutamic acid), or an amino acid sequence having at least 50%, 60%, 65%, 70%, 75%, 80%, 85%, 90%, 95%, 98%, or 99% homology to SEQ ID NO: 10.

Amyloid beta (Amyloid β or Aβ) is a relatively small protein (peptide) composed of approximately 40 amino acid residues and is known to accumulate in the brains of Alzheimer's disease patients, forming amyloid plaques. When amyloid beta accumulates outside neurons in the brain, it induces accumulation of tau protein within neurons. Aggregation of tau protein inside neurons forms neurofibrillary tangles (NFT). The toxicity of accumulated amyloid beta and the accumulation of NFTs are believed to trigger neuronal death, leading to the onset of Alzheimer's dementia.

In addition to Alzheimer's dementia, Lewy body dementia, vascular dementia, and frontotemporal dementia account for approximately 90% of all dementia cases, collectively referred to as the “four major dementias.” While Lewy body dementia, vascular dementia, and frontotemporal dementia are believed to arise through mechanisms distinct from Alzheimer's dementia, there are reports suggesting that amyloid beta accumulation is also associated with brain atrophy in Lewy body dementia.

Diabetes is known to increase the risk of developing Alzheimer's dementia. In patients with diabetes who exhibit increased insulin resistance, insulin secretion is enhanced to lower blood glucose levels. The secreted insulin is degraded by insulin-degrading enzyme (IDE), which also plays a role in degrading amyloid beta. When insulin is present in excess, IDE is consumed for insulin degradation, reducing its ability to degrade amyloid beta. As a result, the undigested amyloid beta is thought to accumulate.

It is also known that individuals with Down syndrome have a relatively high risk of developing early-onset Alzheimer's disease. Down syndrome is a trisomy of chromosome 21, where the gene encoding amyloid precursor protein (APP), the precursor to amyloid beta, is located. Consequently, individuals with Down syndrome produce more amyloid beta compared to healthy individuals, which is considered a cause of their increased risk for developing early-onset Alzheimer's disease.

Diseases caused by the accumulation of amyloid proteins are referred to as amyloidosis. Amyloidosis can be classified into systemic amyloidosis, where amyloid accumulates in multiple organs causing damage, and localized amyloidosis, where amyloid accumulation causes damage in a single organ. Alzheimer's dementia, believed to result from the accumulation of amyloid beta, is a type of localized amyloidosis.

The inventors of the present invention confirmed that adding amyloid beta to neuronal cells results in neuronal cell death, thereby verifying the toxic effects of amyloid beta on neurons. Furthermore, they discovered for the first time that when MANF, APLN, GDF11, TIMP2, THBS4, or CXCL4 is simultaneously added with amyloid beta to neuronal cells, neuronal cell death is suppressed. Therefore, MANF, APLN, GDF11, TIMP2, THBS4, and CXCL4 can each be used as inhibitors of protein accumulation, aggregation, or mislocalization, preferably inhibitors of amyloid beta. Here, the inhibition of amyloid beta includes inhibiting its accumulation, aggregation, or mislocalization, as well as inhibiting its effects, particularly its neurotoxic effects on neurons.

As described above, the accumulation and aggregation of amyloid beta are believed to lead to the onset of Alzheimer's dementia. Therefore, MANF, APLN, GDF11, TIMP2, THBS4, and CXCL4 can each be used as therapeutic or preventive agents for dementia. Dementia may include Alzheimer's dementia and/or Lewy body dementia. Dementia may also include dementia in diabetic patients or in individuals with Down syndrome.

Since the presence of MANF, APLN, GDF11, TIMP2, THBS4, and CXCL4 suppresses neuronal cell death, these proteins can each be used as inhibitors of neuronal cell death or as therapeutic or preventive agents for neurological diseases. Neurological diseases may include those associated with protein accumulation, aggregation, or mislocalization, such as amyloid beta, and preferably those caused by the accumulation or aggregation of amyloid beta, including, for example, neurofibrillary tangles (NFT).

Because MANF, APLN, GDF11, TIMP2, THBS4, and CXCL4 each inhibit amyloid beta, they can be used as therapeutic or preventive agents for amyloidosis. Amyloidosis may be systemic or localized and may include, for example, Alzheimer's dementia.

The inventors discovered for the first time that when GDF11, TIMP2, THBS4, or CXCL4 is added with amyloid beta to neuronal cells, the number of surviving neurons increases compared to when neither amyloid beta nor any of these proteins are added. Therefore, GDF11, TIMP2, THBS4, and CXCL4 can each be used as agents for improving neuronal cell survival rates or as neuroprotective agents.

The inventors also discovered for the first time that when MANF or APLN is added with amyloid beta to neuronal cells, the number of surviving neurons increases compared to when only amyloid beta is added. Therefore, MANF and APLN can each be used as agents for improving neuronal cell survival rates or as neuroprotective agents.

Since MANF, APLN, GDF11, TIMP2, THBS4, and CXCL4 each suppress neuronal cell death or improve neuronal survival rates, they can be used as therapeutic or preventive agents for diseases caused by neuronal cell death. Neuronal cell death includes apoptosis. Diseases caused by neuronal cell death may include Alzheimer's disease, Huntington's disease, Parkinson's disease, and/or amyotrophic lateral sclerosis (ALS). Additionally, MANF, APLN, GDF11, TIMP2, THBS4, and CXCL4 can each be used as therapeutic or preventive agents for the treatment or prevention of neuronal cell loss or neuronal cell death.

The suppression of neuronal cell death, improvement of neuronal survival rates, or protection of neurons contributes to the rejuvenation or prevention of aging in neurons. Therefore, MANF, APLN, GDF11, TIMP2, THBS4, and CXCL4 can each be used as agents for neuronal rejuvenation or anti-aging.

The neuronal cells according to the second embodiment are not limited and may include, for example, cerebral neurons, excitatory neurons, inhibitory neurons, motor neurons, peripheral neurons, and interneurons. The neuronal cells may be harvested neuronal cells or neuronal cells derived from stem cells. Stem cells may include, for example, induced pluripotent stem (iPS) cells. Harvested or differentiated neuronal cells may test positive for proteins such as vGlu (vesicular glutamate transporter), satb2 (Special AT-rich sequence-binding protein 2), CTIP (CtBP-interacting protein), and/or reelin (REELIN).

The pharmaceutical composition and/or cosmetic composition according to the second embodiment comprises an effective amount of MANF, APLN, CXCL4, GDF11, THBS4, and/or TIMP2. Here, “effective amount” refers to an amount sufficient to exert the intended therapeutic or cosmetic effect. The effective amount may be appropriately set based on factors such as the patient's age, the target disease, the presence of other active ingredients, and the quantity of other components.

The pharmaceutical composition and/or cosmetic composition according to the second embodiment may include the pharmaceutically acceptable carriers, excipients, disintegrants, buffers, emulsifiers, suspending agents, anesthetics, stabilizers, preservatives, antimicrobial agents, and physiological saline described in the first embodiment. Additionally, it may include water, alcohol, surfactants, humectants, thickeners, waxes, petrolatum, saturated hydrocarbons, unsaturated fatty acids, silicone oils, and their derivatives, triglycerides, ester oils, natural oils and fats, preservatives, bactericides, UV absorbers, anti-inflammatory agents, antioxidants, chelating agents, plant and animal extracts, pH adjusters, vitamins, fermentation liquids from yeast, koji mold, or lactic acid bacteria, Galactomyces culture fluids, whitening agents, ceramides and ceramide derivatives, anti-wrinkle agents, titanium oxide, talc, mica, silica, zinc oxide, iron oxide, silicone, and processed powders thereof.

Another aspect of the second embodiment is the use of MANF, APLN, CXCL4, GDF11, THBS4, and/or TIMP2 in the manufacture of pharmaceutical compositions and/or cosmetic compositions. Such compositions may include, for example: inhibitors of protein accumulation, aggregation, or mislocalization (preferably inhibitors of amyloid beta); therapeutic or preventive agents for neurological diseases; therapeutic or preventive agents for dementia; therapeutic or preventive agents for diseases caused by neuronal cell death; therapeutic or preventive agents for neuronal cell loss or death; therapeutic or preventive agents for amyloidosis; and/or agents for neuronal rejuvenation or anti-aging.

In the present embodiment, the manufacturing process of a pharmaceutical composition and/or a cosmetic composition may include a step of adding MANF, APLN, CXCL4, GDF11, THBS4, and/or TIMP2. The timing and method of adding MANF, APLN, CXCL4, GDF11, THBS4, and/or TIMP2 are not limited. This embodiment may, for example, include a step of mixing MANF, APLN, CXCL4, GDF11, THBS4, and/or TIMP2 with pharmaceutically acceptable carriers, excipients, disintegrants, buffers, emulsifiers, suspending agents, analgesics, stabilizers, preservatives, or isotonic solutions.

Additionally, the embodiment may include a step of mixing MANF, APLN, CXCL4, GDF11, THBS4, and/or TIMP2 with water, alcohol, surfactants, humectants, thickeners, waxes, petrolatum, saturated and unsaturated fatty acids, silicone oils and their derivatives, triglycerides, ester oils, natural fats and oils, preservatives, antimicrobial agents, ultraviolet absorbers, anti-inflammatory agents, antioxidants, chelating agents, plant and animal extracts, pH adjusters, vitamins, fermentation products of yeast, koji mold, and lactic acid bacteria, Galactomyces ferment filtrate, whitening agents, ceramides and ceramide derivatives, anti-wrinkle agents, titanium oxide, talc, mica, silica, zinc oxide, iron oxide, silicon, and processed powders of these substances. One or more of MANF, APLN, CXCL4, GDF11, THBS4, and TIMP2 may be added. When two or more of MANF, APLN, CXCL4, GDF11, THBS4, and TIMP2 are added, the order of addition is not limited.

Another aspect of the second embodiment pertains to a method of administering a pharmaceutical composition and/or a cosmetic composition comprising MANF, APLN, CXCL4, GDF11, THBS4, and/or TIMP2 to a subject. This aspect may include, for example, methods for inhibiting the accumulation, aggregation, or mislocalization of proteins, preferably amyloid beta; methods for suppressing neuronal cell death; methods for treating or preventing neurological disorders; methods for treating or preventing dementia; methods for treating or preventing diseases caused by neuronal cell death; methods for treating or preventing neuronal cell number reduction or neuronal cell death; methods for treating or preventing amyloidosis; and/or methods for rejuvenating neurons or preventing neuronal aging.

The neurological disorders targeted by this aspect may include disorders associated with the accumulation, aggregation, or mislocalization of proteins such as amyloid beta, preferably neurological disorders caused by amyloid beta accumulation. The neurological disorders may include neurofibrillary tangles (NFT). The dementia targeted by this aspect may include Alzheimer's-type dementia and/or Lewy body dementia. The diseases caused by neuronal cell death targeted by this aspect may include Alzheimer's disease, Huntington's disease, Parkinson's disease, and/or amyotrophic lateral sclerosis (ALS). The amyloidosis targeted by this aspect may include systemic amyloidosis or localized amyloidosis.

In the method according to the second embodiment, the pharmaceutical composition and/or cosmetic composition comprising MANF, APLN, CXCL4, GDF11, THBS4, and/or TIMP2 is administered to a subject. The subject may be a human, a non-human animal, or their cells. The subject may include a human patient with diabetes or a human individual with Down syndrome.

In the method according to the second embodiment, the term “administration” refers to an action similar to the administration described in the method according to the first embodiment.

In the method according to the second embodiment, the pharmaceutical composition and/or cosmetic composition comprising MANF, APLN, CXCL4, GDF11, THBS4, and/or TIMP2 is administered to the subject so that an effective amount of MANF, APLN, CXCL4, GDF11, THBS4, and/or TIMP2 is provided to the subject. Here, the term “effective amount” refers to an amount sufficient to achieve the intended effect as a pharmaceutical or cosmetic composition. The effective amount is appropriately determined based on factors such as the subject's age, the disease condition, the purpose of administration, the presence of other active ingredients, and the quantities of other components.

In the method according to the second embodiment, the frequency, number of times, duration, and other conditions for administering the pharmaceutical composition and/or cosmetic composition comprising MANF, APLN, CXCL4, GDF11, THBS4, and/or TIMP2 to the subject are not limited. These conditions are appropriately adjusted to ensure that an effective amount of MANF, APLN, CXCL4, GDF11, THBS4, and/or TIMP2 is provided to the subject. The frequency of administration of the pharmaceutical composition and/or cosmetic composition comprising MANF, APLN, CXCL4, GDF11, THBS4, and/or TIMP2 may be, for example, twice daily, once daily, once every two days, or once every three days.

### EXAMPLES

### Example 1: Preparation of Test Substances

As the test substance used in Examples 2-5 and 8 described below, recombinant human MANF protein (PEPROTECH) was prepared.

As a positive control substance for Example 2, isoproterenol (Cayman), known to have lipolytic activity, was prepared. For Example 3, insulin (Thermo Fisher Scientific), known to induce differentiation from myoblasts to myotubes, was prepared as a positive control substance. For Example 4, arachidonic acid (Sigma-Aldrich, A3611), known to promote the production of prostacyclin, was prepared as a positive control substance. For Example 5, N-acetylglucosamine (Sigma-Aldrich, A8625), known to promote the production of hyaluronic acid, was prepared as a positive control substance.

For Example 7, the following test substances were prepared:
Recombinant human/mouse/rat GDF11 protein (PEPROTECH) as GDF11.
Recombinant human TIMP2 protein (PEPROTECH) as TIMP2.
Recombinant mouse THBS4 protein (PEPROTECH) as THBS4.
Recombinant human CXCL4 protein (PEPROTECH) as CXCL4.

For Example 8, [Pyr1]-Apelin-13 (CAS No. 217082-60-5) was prepared as APLN. Additionally, recombinant human amyloid beta protein (CAS No. 107761-42-2) was prepared as amyloid beta for use in Examples 7 and 8.

### Example 2: Lipolysis Assay

Using proliferation medium (PGM-2 supplemented with 10% FBS, 2 mM L-glutamine, 50 μg/mL gentamicin, and 37 ng/mL amphotericin B, Lonza, PT-8002), normal human subcutaneous preadipocytes (Lonza, PT-5020) were seeded in T-75 flasks and cultured in a CO₂ incubator (37° C., 5% CO₂, humidified atmosphere, same hereafter) until 60%-80% confluency was achieved. The cells were then detached using 0.25% trypsin-EDTA and centrifuged. The cells were resuspended in proliferation medium and seeded in 96-well plates at a density of 10,000 cells/100 pL/well, followed by incubation in a CO₂ incubator.

The next day, the medium was replaced with 100 pL differentiation medium (PGM-2 supplemented with insulin, dexamethasone, indomethacin, and isobutylmethylxanthine, Lonza, PT-8002), and the cells were cultured in a CO₂ incubator for 7 days to induce differentiation from preadipocytes to adipocytes.

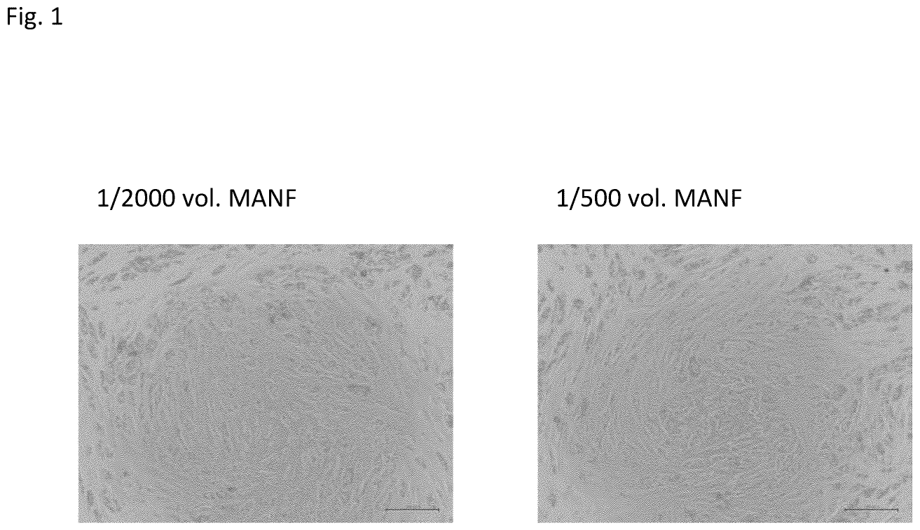

Subsequently, the medium was replaced with 100 μL differentiation medium containing MANF at final dilutions of 1/2000 and 1/500, or the positive control substance at a final concentration of 10 μmol/L. Alternatively, the medium contained neither MANF nor the positive control substance. The cells were incubated in a CO₂ incubator for 24 hours, during which adipocytes were treated with either MANF or the positive control substance. Photographs of the treated cells are shown in FIG. 1.

The supernatant was then collected, and the amount of free glycerol in the supernatant, corresponding to the degree of lipolysis, was measured using a lipolysis assay kit (Cayman, 10009381). The measurement protocol followed the instructions provided with the kit. Specifically, absorbance at a wavelength of 540 nm was measured using a multispectral microplate reader (Varioskan Flash, Thermo Fisher Scientific), and the relative free glycerol concentration was calculated. The group without MANF addition was used as the control. A statistical comparison between the control group and the groups treated with MANF or the positive control substance was conducted using Dunnett's two-tailed test. A p-value of less than 0.05 was considered statistically significant.

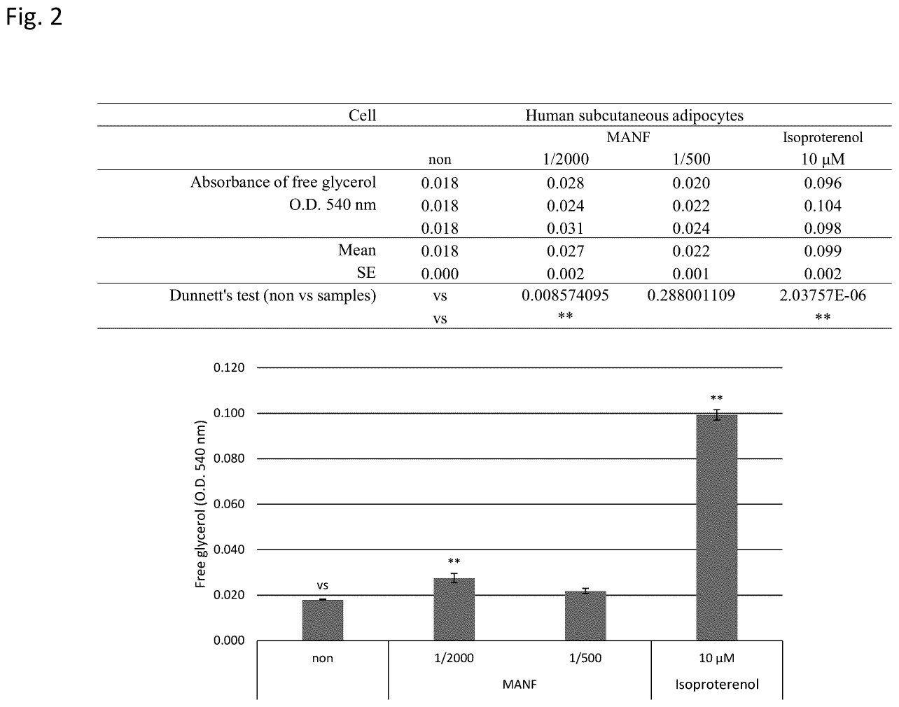

When the final concentration of MANF was 1/2000 by volume, the group treated with MANF exhibited significantly higher lipolysis compared to the control group, as shown in FIG. 2. Similarly, when the final concentration of MANF was 1/500 by volume, the lipolysis level was higher compared to the control group. The group treated with isoproterenol, a substance known to possess lipolytic activity, also exhibited significantly higher lipolysis than the control group, confirming the validity of the experimental system. These results indicate that MANF promotes lipolysis (fat decomposition).

### Example 3: Myotube Differentiation Assay

Using proliferation medium (SkGM-2, Lonza, CC-3245), normal human skeletal muscle myoblasts (Lonza, CC-2580) were seeded into T-75 flasks and cultured in a CO₂ incubator until approximately 60% confluency was achieved. The cells were then detached using 0.05% trypsin-EDTA and phenol red.

Subsequently, the cells were resuspended in proliferation medium and seeded into 96-well plates at a density of 7,500 cells/100 μL/well and incubated in a CO₂ incubator.

The following day, the medium was replaced with 100 μL of differentiation medium (DMEM:F12 supplemented with 2% horse serum) containing MANF at final dilutions of 1/2000 and 1/500, or the positive control substance at a final concentration of 50 nmol/L. Alternatively, the differentiation medium contained neither MANF nor the positive control substance. The cells were then incubated in a CO₂ incubator for 3 days to induce differentiation from myoblasts to myotubes.

Subsequently, the culture medium was removed from the well, and 100 μL of 4% paraformaldehyde at 4° C. was added to the well. The cells were fixed by incubating at 4° C. for 15 minutes. Next, the well was washed three times with 100 μL of Dulbecco's PBS. Following the washing step, 100 μL of a blocking/permeabilization solution (Dulbecco's PBS supplemented with 3% bovine serum albumin and 0.3% Triton X-100) was added to the well, and the mixture was incubated at room temperature for 30 minutes.

The blocking solution was replaced with a solution containing an anti-myosin heavy chain (MHC) antibody (Affymetrix) and incubated overnight at 4° C. Myosin heavy chain is a marker of myotube differentiation. After washing the wells three times with buffer, secondary antibody (goat anti-mouse IgG2b conjugated with Alexa Fluor 555, Thermo Fisher Scientific) and a nuclear staining dye (Hoechst 33342, Thermo Fisher Scientific) were added to the wells and incubated at room temperature for 2 hours.

Following incubation, the wells were washed three times with buffer. Afterward, buffer was added to the wells, and images were captured using a confocal imaging system (Operetta CLS, Perkin Elmer). Nine fields of view at the center of each well were captured using a 10× objective lens.

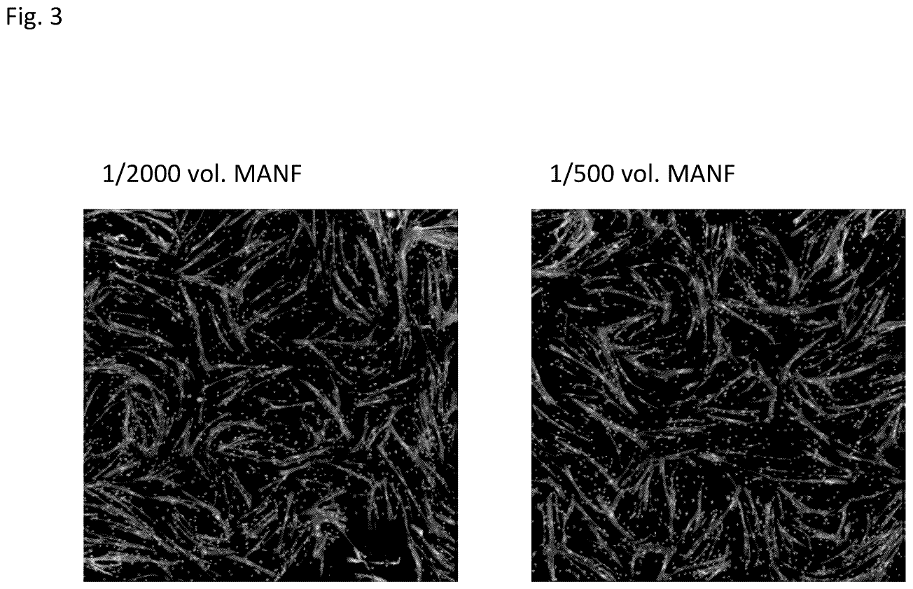

The captured images are shown in FIG. 3. From the captured images, the number of cell nuclei and the number of nuclei positive for myosin heavy chain were measured. Subsequently, a differentiation index I (%) was calculated using Equation (1) below. A higher differentiation index I indicates enhanced myotube differentiation.

I=N_{M}/N_{T}×100 (I)

In Equation (1), N_{M} represents the number of nuclei positive for myosin heavy chain, and N_{T} represents the total number of nuclei.

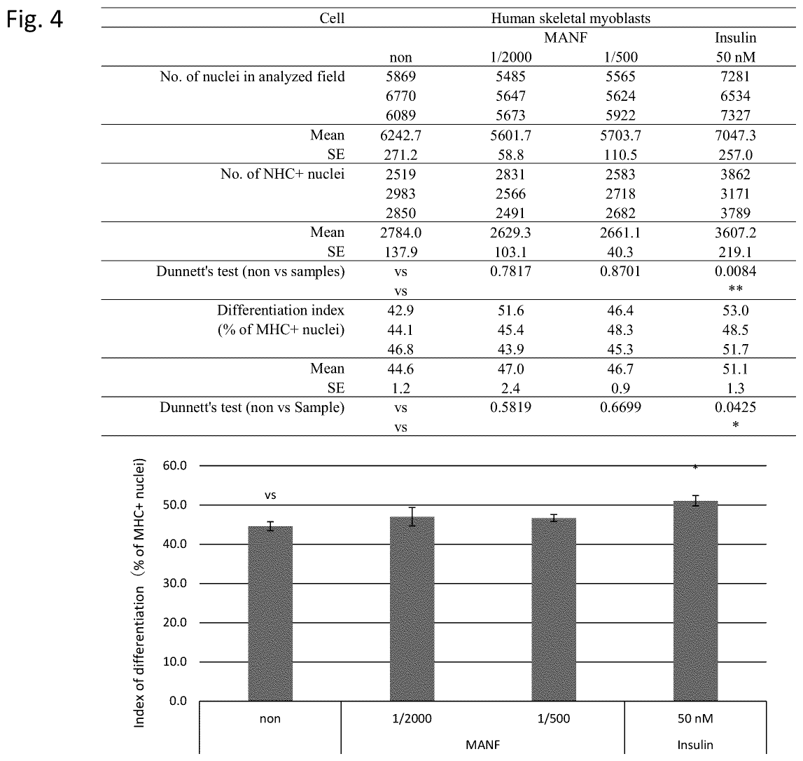

When the final concentration of MANF was 1/2000 by volume, the differentiation index of the MANF-treated group was higher than that of the control group, as shown in FIG. 4. Similarly, when the final concentration of MANF was 1/500 by volume, the differentiation index of the MANF-treated group was also higher compared to the control group. The group treated with insulin, known to induce differentiation from myoblasts to myotubes, exhibited a significantly higher differentiation index compared to the control group, confirming the validity of the experimental system. These results demonstrate that MANF promotes differentiation from normal human skeletal muscle myoblasts to myotubes, enhances myotube proliferation, and/or improves myotube survival rates.

### Example 4: Prostacyclin Production Assay

Using culture medium (EGM-2, Lonza, CC-3162), normal human umbilical vein endothelial cells (HUVECs, Lonza, CC2519AS) were seeded into T-75 flasks and cultured in a CO₂ incubator until 60%-80% confluency was achieved. The cells were then detached using 0.05% trypsin-EDTA and phenol red.

Subsequently, the cells were resuspended in culture medium and seeded into 96-well plates at a density of 10,000 cells/100 pL/well and incubated in a CO₂ incubator.

The next day, the medium was replaced with 100 μL of medium containing MANF at final dilutions of 1/2000 and 1/500, or the positive control substance at a final concentration of 10 μmol/L. Alternatively, the medium contained neither MANF nor the positive control substance. The cells were then incubated in a CO₂ incubator for 24 hours.

The supernatant was collected, and the amount of 6-keto-prostaglandin Fla, a metabolite of prostacyclin, was measured using an ELISA kit (abcam, ab133023) and a multispectral microplate reader (Varioskan Flash, Thermo Fisher Scientific).

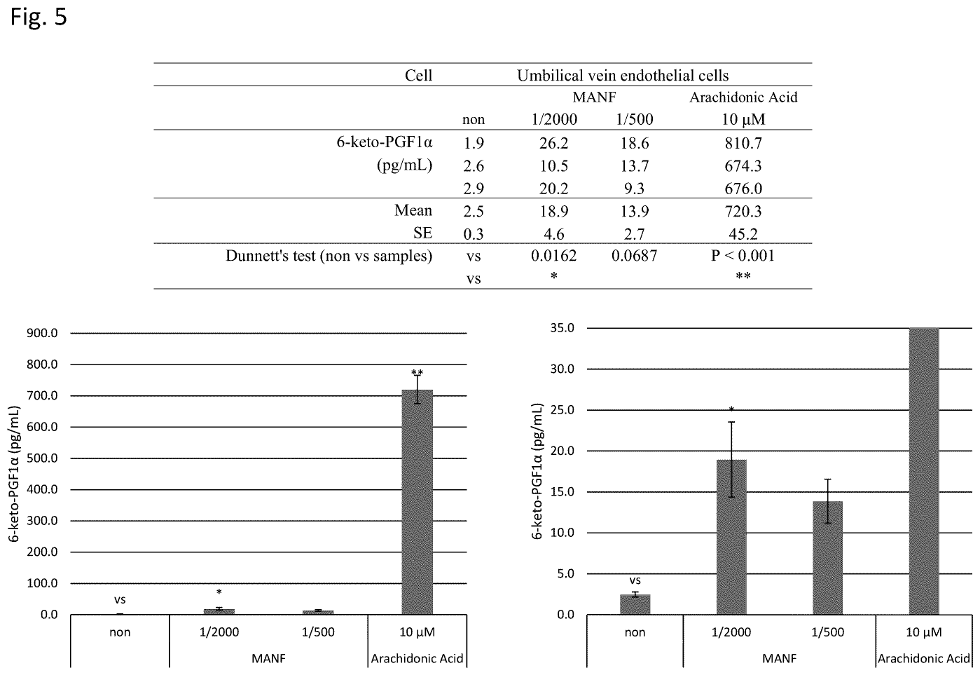

When the final concentration of MANF was 1/2000 by volume, the amount of 6-keto-prostaglandin Fla in the MANF-treated group was significantly higher than in the control group, as shown in FIG. 5. Similarly, when the final concentration of MANF was 1/500 by volume, the MANF-treated group exhibited a higher amount of 6-keto-prostaglandin F1α compared to the control group. The group treated with arachidonic acid, known to promote prostacyclin production, exhibited a significantly higher amount of 6-keto-prostaglandin F1α compared to the control group, confirming the validity of the experimental system. These results demonstrate that MANF promotes the production of prostacyclin by normal human umbilical vein endothelial cells.

### Example 5: Hyaluronic Acid Production Assay

Using proliferation medium (DMEM/F-12 supplemented with 10% FBS and 1% penicillin-streptomycin solution), human chondrocyte cells (SW1353, ATCC, HTB-94) were seeded into T-75 flasks and cultured in a CO₂ incubator until 60%-80% confluency was achieved. The cells were then detached using 0.25% trypsin-EDTA and phenol red. Subsequently, the cells were resuspended in proliferation medium and seeded into 96-well plates at a density of 10,000 cells/100 μL/well and incubated in a CO₂ incubator.

The next day, the medium was replaced with 100 μL of assay medium (DMEM/F-12 supplemented with 1% FBS and 1% penicillin-streptomycin solution) containing MANF at final dilutions of 1/2000 and 1/500, or the positive control substance at a final concentration of 10 mmol/L. Alternatively, the assay medium contained neither MANF nor the positive control substance. The cells were then incubated in a CO₂ incubator for 24 or 72 hours.

The supernatant was collected, and the amount of hyaluronic acid was measured using an ELISA kit (Hyaluronan Quantikine ELISA Kit, R&D Systems) and a multispectral microplate reader (Varioskan Flash, Thermo Fisher Scientific).

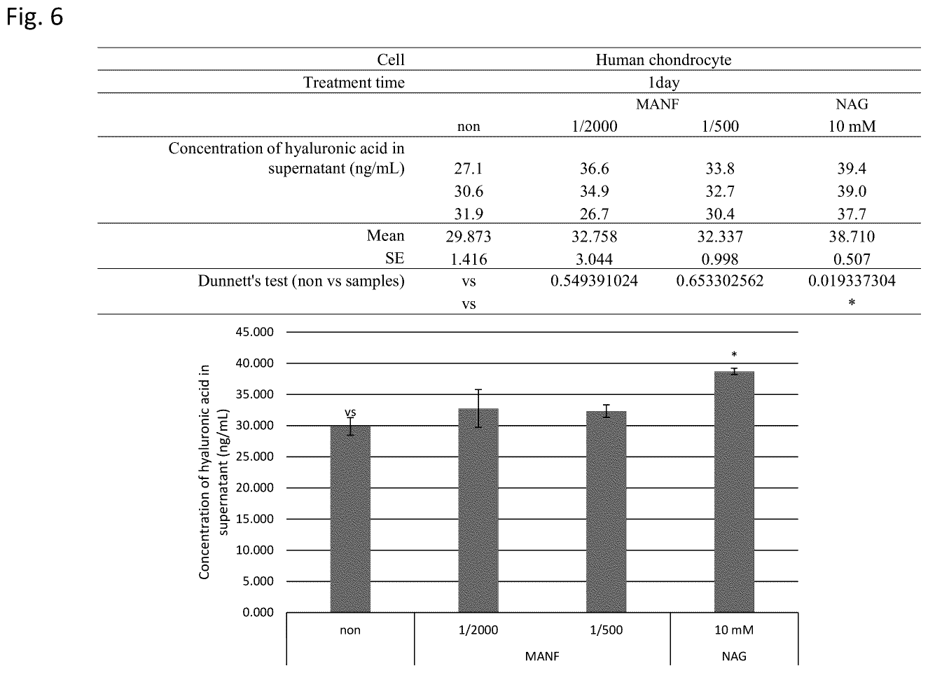

When human chondrocytes were cultured for 24 hours with a final concentration of MANF at 1/2000 by volume, the amount of hyaluronic acid produced in the MANF-treated group was higher than that in the control group, as shown in FIG. 6. Similarly, when MANF was added at a final concentration of 1/500 by volume, the amount of hyaluronic acid produced in the MANF-treated group was higher compared to the control group. The group treated with N-acetylglucosamine, a substance known to promote hyaluronic acid production, exhibited significantly higher hyaluronic acid production compared to the control group, confirming the validity of the experimental system. These results indicate that MANF promotes hyaluronic acid production by human chondrocyte cells.

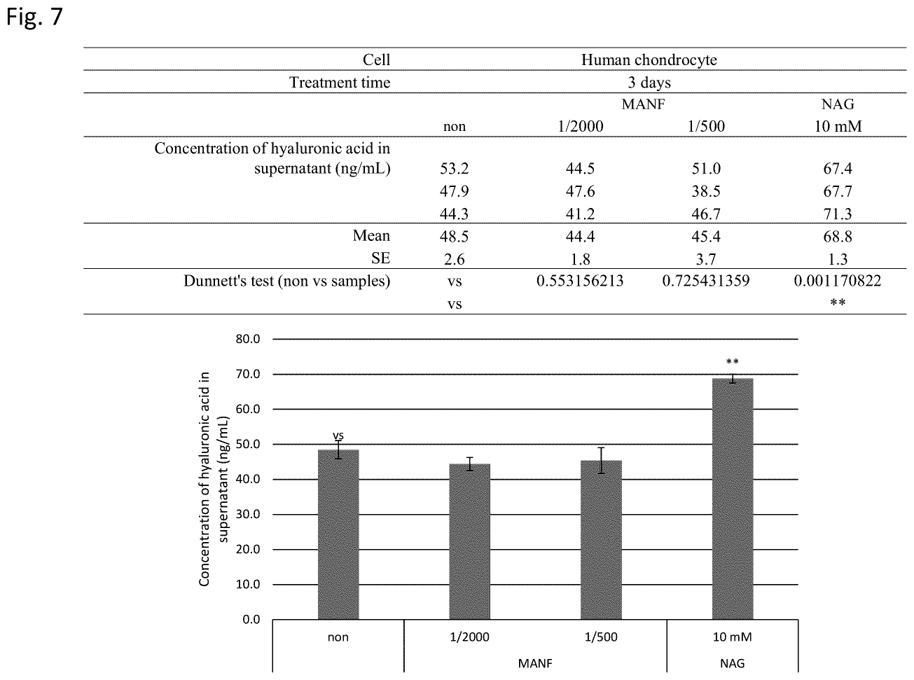

When human chondrocytes were cultured for 72 hours with a final concentration of MANF at 1/2000 by volume, the amount of hyaluronic acid produced in the MANF-treated group was lower than in the control group, as shown in FIG. 7. Similarly, when MANF was added at a final concentration of 1/500 by volume, the amount of hyaluronic acid produced in the MANF-treated group was lower compared to the control group. In contrast, the group treated with N-acetylglucosamine, known to promote hyaluronic acid production, exhibited significantly higher hyaluronic acid production compared to the control group, confirming the validity of the experimental system.

### Example 6: Generation of Cerebral Neurons

A 12-well plate coated with solubilized basement membrane preparation (Matrigel, Corning) was prepared. Each well was filled with feeder-free medium (mTeSR™1, Stemcell Technologies) containing a ROCK (Rho-associated coiled-coil forming kinase) inhibitor at a concentration of 10 nmol/mL (Selleck).

iPS cells were dissociated using a tissue/cell dissociation solution (Accutase, Innovative Cell Technologies) and seeded at a density of 1×10⁵ cells per well into the 12-well plate. The iPS cells were then cultured in feeder-free medium under gas conditions of 5% carbon dioxide and 20% oxygen for 24 hours.

The next day (Day 0), RNA encoding NGN2 (neurogenin 2) linked to RNA corresponding to a puromycin resistance gene was introduced into the iPS cells. On the second day after NGN2-puromycin resistance gene introduction (Day 2), the medium in the wells was replaced with neural induction medium (N3 medium) containing puromycin at a concentration of 2 μg/mL. The cells were cultured for an additional 3 days to eliminate non-NGN2-transduced iPS cells and select NGN2-transduced iPS cells. The N3 medium was prepared by adding 10 mL of B27, 5 mL of N2, and 1.6 mL of insulin at a concentration of 6.25 mg/mL to 500 mL of DMEM/F12.

On the seventh day after NGN2-puromycin resistance gene introduction (Day 7), it was confirmed that cerebral neurons positive for vGLU, SATB2, CTIP, and REELIN were induced from the iPS cells.

### Example 7: Survival Assay of Cerebral Neurons Using GDF11, TIMP2, THBS4, and CXCL4

On Day 7 following the introduction of the NGN2-puromycin resistance gene (Day 7), after confirming the induction of cerebral neurons from iPS cells, recombinant amyloid beta protein (10 μM) and one of GDF11, TIMP2, THBS4, or CXCL4 were simultaneously added to the neural induction medium, and the cerebral neurons were cultured.

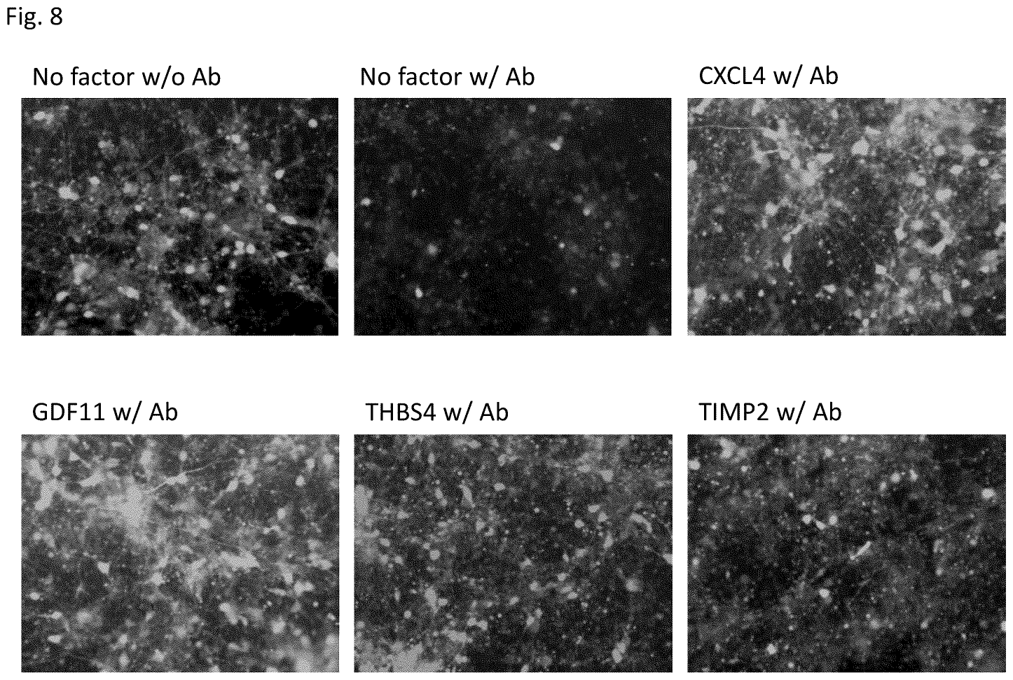

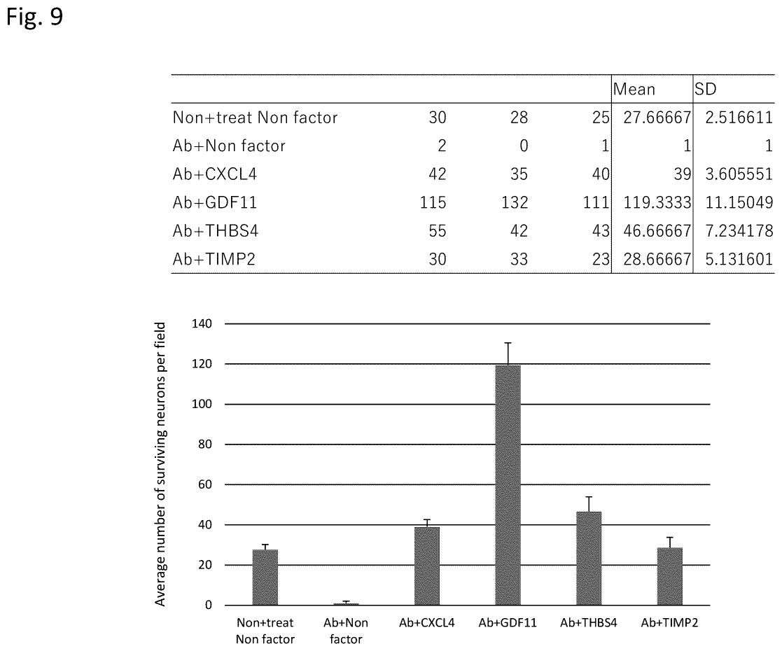

As a first control group (No factor w/o Ab), cerebral neurons were cultured without amyloid beta, GDF11, TIMP2, THBS4, or CXCL4. As a second control group (No factor w/Ab), cerebral neurons were cultured with amyloid beta (10 μM) alone. The medium was replaced every five days. On Day 17 following NGN2-puromycin resistance gene introduction (Day 17), the number of surviving cerebral neurons per field was measured in five fields and averaged. Examples of images of the measured fields are shown in FIG. 8, and the averaged number of surviving neurons per field is shown in FIG. 9.

As shown in FIGS. 8 and 9, the number of surviving cerebral neurons per field in the second control group (No factor w/Ab) was lower than that in the first control group (No factor w/o Ab). These results demonstrate that amyloid beta induces the death of cerebral neurons.

As shown in FIGS. 8 and 9, the groups in which amyloid beta and one of GDF11, TIMP2, THBS4, or CXCL4 were simultaneously added to the cerebral neurons exhibited a higher number of surviving neurons per field compared to the second control group (No factor w/Ab). These results indicate that GDF11, TIMP2, THBS4, and CXCL4 each inhibit the effects of amyloid beta and suppress amyloid beta-induced neuronal death. Furthermore, the groups treated with amyloid beta and one of GDF11, TIMP2, THBS4, or CXCL4 showed an increase in the number of surviving cerebral neurons per field compared to the first control group (No factor w/o Ab).

### Example 8: Survival Assay of Cerebral Neurons Using MANF and APLN

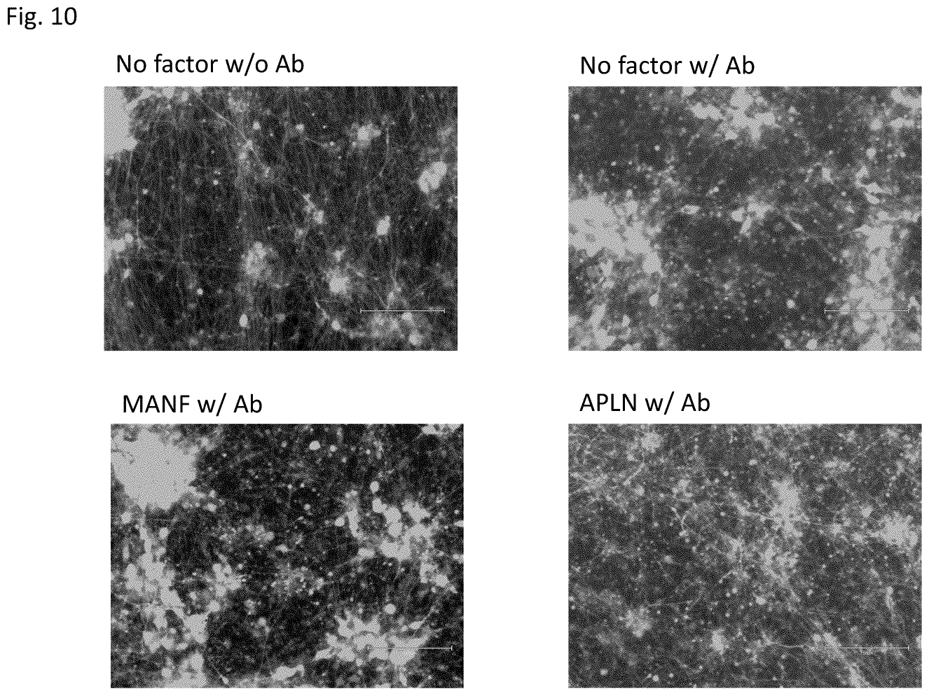

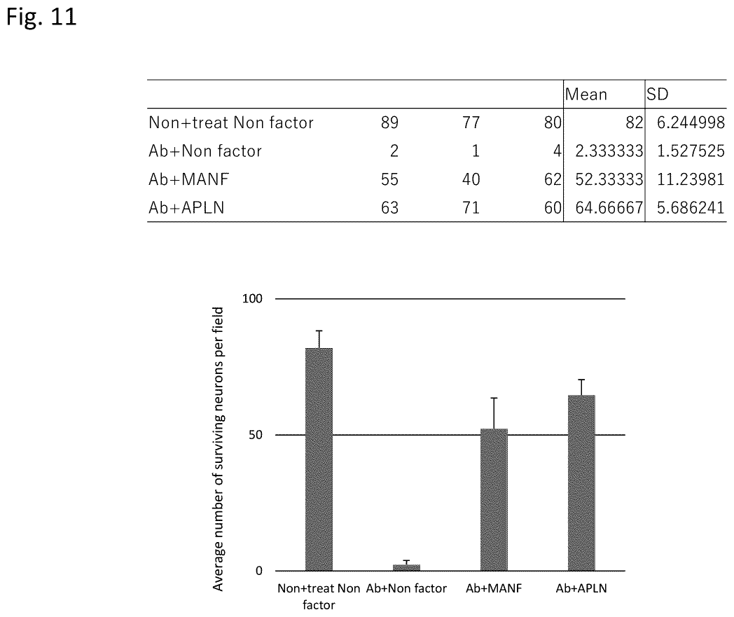

The experiment was conducted similarly to Example 7, except that MANF and APLN were used instead of GDF11, TIMP2, THBS4, and CXCL4. Examples of images of the measured fields are shown in FIG. 10, and the averaged number of surviving neurons per field is shown in FIG. 11.

As shown in FIGS. 10 and 11, the number of axons and surviving cerebral neurons in the second control group (No factor w/Ab), where amyloid beta (10 μM) alone was added, were lower than those in the first control group (No factor w/o Ab), where no amyloid beta, MANF, or APLN was added. These results confirm that amyloid beta induces the death of cerebral neurons, consistent with the results obtained in Example 7.

As shown in FIGS. 10 and 11, the groups where amyloid beta and either MANF or APLN were simultaneously added to the cerebral neurons exhibited a greater number of surviving cerebral neurons per field compared to the second control group (No factor w/Ab). These results indicate that both MANF and APLN inhibit the effects of amyloid beta and suppress amyloid beta-induced neuronal death.

### Example 9: Apoptosis Marker Assay

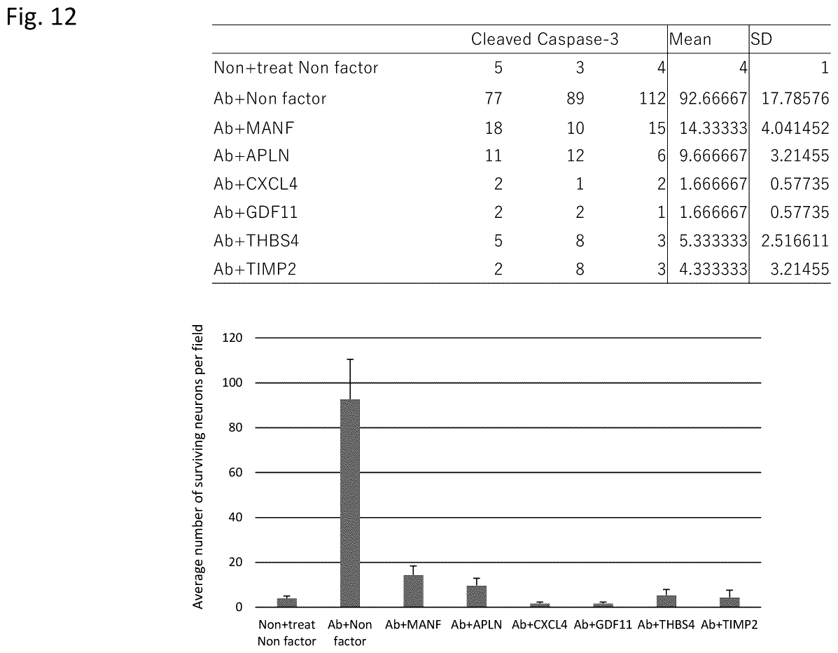

Cerebral neurons cultured in Examples 7 and 8 were stained with an apoptosis marker, anti-Cleaved Caspase-3 antibody (Abcam), at a 1/100 dilution. After staining, the number of Cleaved Caspase-3-positive cerebral neurons per field was measured in five fields and averaged. The average number of Cleaved Caspase-3-positive cerebral neurons per field is shown in FIG. 12.

As shown in FIG. 12, the groups where amyloid beta and one of MANF, APLN, GDF11, TIMP2, THBS4, or CXCL4 were simultaneously added to the cerebral neurons exhibited a reduced number of Cleaved Caspase-3-positive cerebral neurons per field compared to the first control group (No factor w/o Ab).

The results of Examples 7-9 demonstrate that MANF, APLN, GDF11, TIMP2, THBS4, and CXCL4 each suppress neuronal cell death and improve cell survival rates. Notably, in the group where amyloid beta and GDF11 were simultaneously added to the cerebral neurons, the number of surviving cerebral neurons per field increased more than fourfold compared to the first control group (No factor w/o Ab).

These results indicate that, compared to MANF, APLN, TIMP2, THBS4, or CXCL4, GDF11 has a particularly significant effect on enhancing neuronal cell survival rates or protecting neuronal cells from cell death stress or ER stress.

1.-20. (canceled)

21. A method for decomposing fat, comprising:
administering an effective amount of a composition comprising MANF (Mesencephalic astrocyte-derived neurotrophic factor) protein to a subject in need thereof.

22. A method for treating obesity, comprising:
decomposing fat by the method of claim 21.

23. A method for treating diabetes, comprising:
decomposing fat by the method of claim 21.

24. A method for promoting production of prostacyclin or cyclic adenosine monophosphate (cAMP), comprising:
administering an effective amount of a composition comprising MANF (Mesencephalic astrocyte-derived neurotrophic factor) protein to a subject in need thereof.

25. A method for inhibiting aggregation of a platelet, comprising:
promoting production of prostacyclin by the method of claim 24.

26. A method for vasodilation, comprising:
promoting production of prostacyclin by the method of claim 24.

27. A method for treating hypertension or arteriosclerosis, comprising:
promoting production of prostacyclin by the method of claim 24.

28. A method for inhibiting proliferation of a vascular endothelial cell, comprising:
promoting production of prostacyclin by the method of claim 24.

29. A method for promoting production of hyaluronic acid in chondrocytes, comprising:
administering an effective amount of a composition comprising MANF (Mesencephalic astrocyte-derived neurotrophic factor) protein to a subject in need thereof.

30. A method for treating a joint disease or joint pain, comprising:
promoting production of hyaluronic acid by the method of claim 29.

31. A method for inducing differentiation of a myoblast to a myotube, comprising:
administering an effective amount of a composition comprising MANF (Mesencephalic astrocyte-derived neurotrophic factor) protein to a subject in need thereof.

32. A method for promoting formation of muscle, comprising:
inducing differentiation of a myoblast to a myotube by the method of claim 31.

33. A method for maintaining or increasing muscle mass, comprising:
inducing differentiation of a myoblast to a myotube by the method of claim 31.

34. A method for treating a lifestyle-related disease or an adult-onset disease, comprising:
administering an effective amount of a composition comprising MANF (Mesencephalic astrocyte-derived neurotrophic factor) protein to a subject in need thereof.

35. A method for inhibiting accumulation of amyloid beta, comprising:
administering an effective amount of a composition comprising at least one protein selected from the group consisting of MANF (Mesencephalic astrocyte-derived neurotrophic factor), APLN (Apelin), CXCL4 (Chemokine C-X-C Motif Ligand 4), GDF11 (Growth differentiation factor 11), THBS4 (Thrombospondin-4), and TIMP2 (Tissue inhibitor of metalloproteinases 2) to a subject in need thereof.

36. A method for treating a neurological disease, comprising:
inhibiting accumulation of amyloid beta by the method of claim 35.

37. A method for treating dementia, comprising:
inhibiting accumulation of amyloid beta by the method of claim 35.

38. A method for inhibiting neuronal cell death, comprising:
inhibiting accumulation of amyloid beta by the method of claim 35.

39. A method for preventing reduction in a number of neuronal cells, comprising:
inhibiting accumulation of amyloid beta by the method of claim 35.

40. The method according to claim 36, wherein the neurological disease is a disease caused by accumulation of amyloid beta.
